SEQUENCE LISTING (75 sequences)

SEQ ID NO: 1 - DNA, 1461 bp, Staphylococcus bacteriophage 2638A
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 2 - protein, 486 aa, Staphylococcus bacteriophage 2638A
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 3 - DNA, 279 bp, Staphylococcus simulans
tggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtga

SEQ ID NO: 4 - protein, 92 aa, Staphylococcus simulans
WKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 5 - DNA, 285 bp, Staphylococcus bacteriophage 2638A
tggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 6 - protein, 94 aa, Staphylococcus bacteriophage 2638A
WKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 7 - DNA, 282 bp, Staphylococcus bacteriophage phiNM3
ggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttt

SEQ ID NO: 8 - protein, 94 aa, Staphylococcus bacteriophage phiNM3
GKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 9 - DNA, 585 bp, Staphylococcus bacteriophage K
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaat

SEQ ID NO: 10 - protein, 195 aa, Staphylococcus bacteriophage K
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKN

SEQ ID NO: 11 - DNA, 420 bp, Staphylococcus bacteriophage Twort
atgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttatt

SEQ ID NO: 12 - protein, 140 aa, Staphylococcus bacteriophage Twort
MKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFI

SEQ ID NO: 13 - DNA, 438 bp, Staphylococcus bacteriophage 2638A
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagat

SEQ ID NO: 14 - protein, 146 aa, Staphylococcus bacteriophage 2638A
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKD

SEQ ID NO: 15 - DNA, 420 bp, Staphylococcus simulans
gctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatat

SEQ ID NO: 16 - protein, 140 aa, Staphylococcus simulans
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGY

SEQ ID NO: 17 - DNA, 510 bp, Staphylococcus bacteriophage 2638A
ggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggt

SEQ ID NO: 18 - protein, 169 aa, Staphylococcus bacteriophage 2638A
NKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDG

SEQ ID NO: 19 - DNA, 1407 bp, Artificial Sequence
  artificial construct
atggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 20 - protein, 468 aa, Artificial Sequence
  artificial construct
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 21 - DNA, 1209 bp, Artificial Sequence
  artificial construct
gctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 22 - protein, 402 aa, Artificial Sequence
  artificial construct
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 23 - DNA, 1686 bp, Artificial Sequence
  artificial construct
gcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 24 - protein, 561 aa, Artificial Sequence
  artificial construct
AKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 25 - DNA, 1131 bp, Artificial Sequence
  artificial construct
atgagaggatcgcatcaccatcaccatcacggatctggctctggatctggtatcgagggaaggccttataatggaactggatccgcatgcgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 26 - protein, 376 aa, Artificial Sequence
  artificial construct
MRGSHHHHHHGSGSGSGIEGRPYNGTGSACELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 27 - DNA, 1125 bp, Artificial Sequence
  artificial construct
atgagaggatcgcatcaccatcaccatcacggatctggctctggatctggtatcgagggaaggccttataatggaactggatccgcatgcgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 28 - protein, 374 aa, Artificial Sequence
  artificial construct
MRGSHHHHHHGSGSGSGIEGRPYNGTGSACELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 29 - DNA, 1131 bp, Artificial Sequence
  artificial construct
atgagaggatcgcatcaccatcaccatcacggatctggctctggatctggtatcgagggaaggccttataatggaactggatccgcatgcgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 30 - protein, 376 aa, Artificial Sequence
  artificial construct
MRGSHHHHHHGSGSGSGIEGRPYNGTGSACELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 31 - DNA, 975 bp, Artificial Sequence
  artificial construct
atgagaggatcgcatcaccatcaccatcacggatctggctctggatctggtatcgagggaaggccttataatggaactggatccgcatgcgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 32 - protein, 324 aa, Artificial Sequence
  artificial construct
MRGSHHHHHHGSGSGSGIEGRPYNGTGSACELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 33 - DNA, 912 bp, Artificial Sequence
  artificial construct
atgagaggatcgcatcaccatcaccatcacggatctggctctggatctggtatcgagggaaggccttataatggaactggatccgcatgcgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 34 - protein, 303 aa, Artificial Sequence
  artificial construct
MRGSHHHHHHGSGSGSGIEGRPYNGTGSACELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 35 - DNA, 966 bp, Artificial Sequence
  artificial construct
atgagaggatcgcatcaccatcaccatcacggatctggctctggatctggtatcgagggaaggccttataatggaactggatccgcatgcgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 36 - protein, 321 aa, Artificial Sequence
  artificial construct
MRGSHHHHHHGSGSGSGIEGRPYNGTGSACELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 37 - DNA, 927 bp, Artificial Sequence
  artificial construct
atgagaggatcgcatcaccatcaccatcacggatctggctctggatctggtatcgagggaaggccttataatggaactggatccgcatgcgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 38 - protein, 308 aa, Artificial Sequence
  artificial construct
MRGSHHHHHHGSGSGSGIEGRPYNGTGSACELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 39 - DNA, 921 bp, Artificial Sequence
  artificial construct
atgagaggatcgcatcaccatcaccatcacggatctggctctggatctggtatcgagggaaggccttataatggaactggatccgcatgcgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 40 - protein, 306 aa, Artificial Sequence
  artificial construct
MRGSHHHHHHGSGSGSGIEGRPYNGTGSACELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 41 - DNA, 927 bp, Artificial Sequence
  artificial construct
atgagaggatcgcatcaccatcaccatcacggatctggctctggatctggtatcgagggaaggccttataatggaactggatccgcatgcgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 42 - protein, 308 aa, Artificial Sequence
  artificial construct
MRGSHHHHHHGSGSGSGIEGRPYNGTGSACELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 43 - DNA, 843 bp, Artificial Sequence
  artificial construct
atgagaggatcgcatcaccatcaccatcacggatctggctctggatctggtatcgagggaaggccttataatggaactggatccgcatgcgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 44 - protein, 280 aa, Artificial Sequence
  artificial construct
MRGSHHHHHHGSGSGSGIEGRPYNGTGSACELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 45 - DNA, 837 bp, Artificial Sequence
  artificial construct
atgagaggatcgcatcaccatcaccatcacggatctggctctggatctggtatcgagggaaggccttataatggaactggatccgcatgcgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 46 - protein, 278 aa, Artificial Sequence
  artificial construct
MRGSHHHHHHGSGSGSGIEGRPYNGTGSACELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 47 - DNA, 843 bp, Artificial Sequence
  artificial construct
atgagaggatcgcatcaccatcaccatcacggatctggctctggatctggtatcgagggaaggccttataatggaactggatccgcatgcgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 48 - protein, 280 aa, Artificial Sequence
  artificial construct
MRGSHHHHHHGSGSGSGIEGRPYNGTGSACELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 49 - DNA, 1755 bp, Artificial Sequence
  artificial construct
atgagaggatcgcatcaccatcaccatcacggatctggctctggatctggtatcgagggaagggcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 50 - protein, 584 aa, Artificial Sequence
  artificial construct
MRGSHHHHHHGSGSGSGIEGRAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 51 - DNA, 1749 bp, Artificial Sequence
  artificial construct
atgagaggatcgcatcaccatcaccatcacggatctggctctggatctggtatcgagggaagggcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 52 - protein, 582 aa, Artificial Sequence
  artificial construct
MRGSHHHHHHGSGSGSGIEGRAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 53 - DNA, 1755 bp, Artificial Sequence
  artificial construct
atgagaggatcgcatcaccatcaccatcacggatctggctctggatctggtatcgagggaagggcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtgagctcgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 54 - protein, 584 aa, Artificial Sequence
  artificial construct
MRGSHHHHHHGSGSGSGIEGRAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGELAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 55 - DNA, 1533 bp, Artificial Sequence
  artificial construct
atgagaggatcgcatcaccatcaccatcacggatctggctctggatctggtatcgagggaaggatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 56 - protein, 510 aa, Artificial Sequence
  artificial construct
MRGSHHHHHHGSGSGSGIEGRMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 57 - DNA, 1470 bp, Artificial Sequence
  artificial construct
atgagaggatcgcatcaccatcaccatcacggatctggctctggatctggtatcgagggaaggatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 58 - protein, 489 aa, Artificial Sequence
  artificial construct
MRGSHHHHHHGSGSGSGIEGRMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 59 - DNA, 1524 bp, Artificial Sequence
  artificial construct
atgagaggatcgcatcaccatcaccatcacggatctggctctggatctggtatcgagggaaggatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatgagctcatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 60 - protein, 507 aa, Artificial Sequence
  artificial construct
MRGSHHHHHHGSGSGSGIEGRMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNELMAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 61 - DNA, 1446 bp, Artificial Sequence
  artificial construct
atgagaggatcgcatcaccatcaccatcacggatctggctctggatctggtatcgagggaaggatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 62 - protein, 481 aa, Artificial Sequence
  artificial construct
MRGSHHHHHHGSGSGSGIEGRMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 63 - DNA, 1440 bp, Artificial Sequence
  artificial construct
atgagaggatcgcatcaccatcaccatcacggatctggctctggatctggtatcgagggaaggatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaattggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 64 - protein, 479 aa, Artificial Sequence
  artificial construct
MRGSHHHHHHGSGSGSGIEGRMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 65 - DNA, 1446 bp, Artificial Sequence
  artificial construct
atgagaggatcgcatcaccatcaccatcacggatctggctctggatctggtatcgagggaaggatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatgagctcatgaaaaccctgaaacaagcagagtcctacattaagagtaaagtaaatacaggaactgattttgatggtttatatgggtatcagtgtatggacttagcagtagattatatttaccatgtaacagatggtaaaataagaatgtggggtaatgctaaggatgcgataaataactcttttggtggtactgctacggtatataaaaactaccctgcttttagacctaagtacggtgatgtagtcgtatggactactggtaattttgcaacttatggtcatatcgcaatagttactaaccctgacccttatggagaccttcaatatgttacagttcttgaacaaaactggaacggtaacgggatttataaaaccgagttagctacaatcagaacacacgattacacaggaattacacattttattagacctaactttgctactgaatcaagtgtaaaaaagaaagatacaaagaaaaaaccaaaaccatcaaatagagatggaataaataaagataaaattgtatatgatagaactaatattaattacaatggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 66 - protein, 481 aa, Artificial Sequence
  artificial construct
MRGSHHHHHHGSGSGSGIEGRMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNELMKTLKQAESYIKSKVNTGTDFDGLYGYQCMDLAVDYIYHVTDGKIRMWGNAKDAINNSFGGTATVYKNYPAFRPKYGDVVVWTTGNFATYGHIAIVTNPDPYGDLQYVTVLEQNWNGNGIYKTELATIRTHDYTGITHFIRPNFATESSVKKKDTKKKPKPSNRDGINKDKIVYDRTNINYNGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 67 - DNA, 1278 bp, Artificial Sequence
  artificial construct
atgagaggatcgcatcaccatcaccatcacggatctggctctggatctggtatcgagggaagggctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 68 - protein, 425 aa, Artificial Sequence
  artificial construct
MRGSHHHHHHGSGSGSGIEGRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 69 - DNA, 1272 bp, Artificial Sequence
  artificial construct
atgagaggatcgcatcaccatcaccatcacggatctggctctggatctggtatcgagggaagggctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggttggaaaacaaacaaatatggcacactatataaatcagagtcagctagcttcacacctaatacagatataataacaagaacgactggtccatttagaagcatgccgcagtcaggagtcttaaaagcaggtcaaacaattcattatgatgaagtgatgaaacaagacggtcatgtttgggtaggttatacaggtaacagtggccaacgtatttacttgcctgtaagaacatggaataaatctactaatactttaggtgttctttggggaactataaagtaa

SEQ ID NO: 70 - protein, 423 aa, Artificial Sequence
  artificial construct
MRGSHHHHHHGSGSGSGIEGRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 71 - DNA, 1278 bp, Artificial Sequence
  artificial construct
atgagaggatcgcatcaccatcaccatcacggatctggctctggatctggtatcgagggaagggctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcgctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtggtaaatctgcaagtaaaataacagttggaagtaaagcgccttataaccttaaatggtcaaaaggtgcttattttaatgcgaaaatcgacggcttaggtgctacttcagccactagatacggtgataatcgtactaactatagattcgatgttggacaggctgtatacgcgcctggaacattaatatatgtgtttgaaattatagatggttggtgtcgcatttattggaacaatcataatgagtggatatggcatgagagattgattgtgaaagaagtgttttaa

SEQ ID NO: 72 - protein, 425 aa, Artificial Sequence
  artificial construct
MRGSHHHHHHGSGSGSGIEGRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 73 - protein, 506 aa, Artificial Sequence
  artificial construct
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 74 - protein, 462 aa, Artificial Sequence
  artificial construct
MAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGLKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 75 - protein, 10 aa, Artificial Sequence
  Synthetic peptide
ELAGIGILTV

(19) United States
(12) Patent Application Publication
(10) Pub. No.: US 2020/0291373 A1
(43) Pub. Date: Sep. 17, 2020

(54) Treatment of a Condition Associated with Infection with an Oncogenic Bacterium

(71) Applicant: Micreos Human Health B.V., EV Den Haag (NL)

(72) Inventor: Mark Leonard Offerhaus, SN Den Haag (NL)

(21) Appl. No.: 16/753,162

(22) PCT Filed: Oct. 5, 2018
(86) PCT No.: PCT/EP2018/077155

§ 371 Date: Apr. 2, 2020

(30) Foreign Application Priority Data
Oct. 6, 2017 (EP) 17195324.3

(51) Int. Cl. C12N 9/36; A61P 35/00; A61K 38/48

(52) U.S. Cl. CPC C12N 9/2462; A61P 35/00; A61K 45/06; C12Y 302/01017; A61K 38/4813

(57) ABSTRACT

The present invention relates to the field of medicine, specifically to the field of treatment of a malignant condition associated with infection with a bacterium that aggravates and/or induces proliferation of the malignant conditions.

### FIELD OF THE INVENTION

The present invention relates to the field of medicine, specifically to the field of treatment of a malignant condition associated with infection with a bacterium that aggravates and/or induces proliferation of the malignant conditions.

### BACKGROUND OF THE INVENTION

Oncogenic bacteria can be a major problem in cancer patients. In several types of cancer, bacteria play a role in pathogenesis and/or oncogenesis. Helicobacter pylori infection is e.g. closely related with chronic gastritis, gastric duodenal ulcer, and mucosa associated diseases such as lymphoma and gastric cancer (Nervi G, et al. 2006, Does Helicobacter pylori infection eradication modify peptic ulcer prevalence? A 10 years' endoscopical survey. World J Gastroenterol 12: 2398-2401; Kamangar et al. 2007, Helicobacter pylori and oesophageal and gastric cancers in a prospective study in China. Br J Cancer 96: 172-176). It has been classified as class I carcinogen in 1994 by the World Health Organization and the International Agency for Research on Cancer consensus group (Uemura N, et al. 2001, Helicobacter pylori infection and the development of gastric cancer. N Engl J Med 345: 784-789.).

Recently, Staphylococcal enterotoxin A (SEA) has been demonstrated to stimulates Signal transducer and activator of transcription 3 (STAT3) activation and expression of STAT3 driven effector molecules, such as IL-17 and IL-10, in cutaneous T-cell lymphoma (CTCL) (Willerslev-Olsen, et al. 2016, Staphylococcal enterotoxin A (SEA) stimulates STAT3 activation and IL-17 expression in cutaneous T-cell lymphoma. Blood, 10 Mar. 2016, vol. 127-10; Krejsgaard T, et al., Staphylococcal enterotoxins stimulate lymphoma-associated immune dysregulation; Blood. 2014 Jul. 31; 124(5):761-70). Hence, SEA promotes activation of an established oncogenic pathway previously implicated in carcinogenesis and Staphylococcus aureus can thus be regarded as an oncogenic bacterium according to the definitions herein.

Treatment of these bacterial infections is suggested by use of antibiotics, but antibiotics have the disadvantage that there is factual chance that the target bacteria become resistant (e.g. MRSA). In addition, antibiotics are not very specific and will disturb the balance of commensal benign bacteria; said balance is already fragile and compromised in cancer patients. Moreover, there is no evidence to date that treating CTCL patients with antibiotics has an influence on carcinogenesis. Accordingly, there is a need to a novel method of treatment of cancer patients where oncogenic bacteria are associated with pathogenesis, progression and/or oncogenesis of the cancer.

### BRIEF DESCRIPTION OF THE FIGURES

FIG. 1A: Proliferation of a malignant and a non-malignant (Mycosis Fungoides) patient derived CTCL cell line with and without alpha-toxin from Staphylococcus aureus.

FIG. 1B: Cytotoxicity assay of MART-1 specific CD8 T cells directed against peptide pulsed Mac1 cells with and without alpha-toxin from Staphylococcus aureus.

### DESCRIPTION OF THE INVENTION

It has been established by the inventors that, surprisingly, an endolysin can effectively be used in the treatment of cancer. Without being bound by theory, it is believed that the specificity of the bacteriophage endolysin in killing target bacteria together with its effectiveness in killing target bacteria provides the unexpected superior beneficial effect in the treatment of cancer.

Accordingly, the invention provides for an endolysin specific for an oncogenic bacterium for use in the prevention, delay or treatment of cancer in a subject in need thereof.

In all embodiments of the invention, the terms lysin, endolysin and autolysin are used interchangeably. The endolysin specific for an oncogenic bacterium is herein referred as the endolysin according to the invention. The oncogenic bacterium is herein referred to as the oncogenic bacterium according to the invention. In the context of the invention, an oncogenic bacterium is an oncogenic bacterium according to general knowledge in the field, but also a bacterium that in a malignancy associated with infection with said bacterium, aggravates and/or induces proliferation of the malignant condition.

In all embodiments of the invention, the oncogenic bacterium may be any oncogenic bacterium. It may be a bacterium selected from the group consisting of Staphylococcus, Streptococcus (such as S. pyogenes, S. agalactiae and S. pneumonia), Actinomyces, Nocardia, Bacillus (such as B. anthracis), Coxiella (such as Coxiella burnetii), Rickettsia, Mycobacteria (such as M. tuberculosis and M. leprae), Legionella (such as L. pneumophila), Mycoplasma, Salmonella (such as S. typhimurium), Shigella (such as S. dysenteriae), Yersinia (such as Y. pestis), Brucella, Listeria (such as L. monocytogenesis), Actinobacillus (such as A. actinomycetemcomitans), Gardnerella (such as G. vaginalis), Chlamydia (such as C. trachomatis), and Chlamidophila; a more preferred bacterium is a species of Staphylococcus; a preferred species of Staphylococcus is S. aureus. A preferred S. aureus is an antibiotic resistant Staphylococcus aureus, such as a methicillin resistant Staphylococcus aureus (MRSA).

Preferably, the endolysin according to the invention is a Staphylococcus-specific endolysin, meaning that it will lyse Staphylococcus efficiently but does not substantially lyse other bacteria than Staphylococcus. Most native Staphylococcus bacteriophage endolysins exhibiting peptidoglycan hydrolase activity consist of a C-terminal cell wall-binding domain (CBD), a central N-acetylmuramoyl-L-Alanine amidase domain, and an N-terminal Alanyl-glycyl endopeptidase domain with cysteine, histidine-dependent amidohydrolases/peptidase (CHAP) homology, or in case of Ply2638, of an N-terminal glycyl-glycine endopeptidase domain with Peptidase_M23 homology, the latter three domains exhibiting peptidoglycan hydrolase activity each with distinct target bond specificity and generally named as enzymatically active domains. The Ply2638 endolysin is set forward in SEQ ID NO: 1 and SEQ ID NO: 2 (see Table 1); several endolysin domains are set forward in SEQ ID NO: 3 to SEQ ID NO: 18 (see Table 1), these domains are preferred domains according to the invention.

A more preferred endolysin according to the invention is a recombinant endolysin, more preferably a recombinant Staphylococcus-specific endolysin, even more preferably a recombinant Staphylococcus-specific chimeric endolysin comprising one or more heterologous domains. In general, endolysins are comprised of different subunits (domains); e.g. a cell wall-binding domain (CBD) and one or more enzymatic domains having peptidoglycan activity, such as an amidase domain, an M23 peptidase domain and a CHAP (cysteine, histidine-dependent amidohydrolases/peptidases) domain. An example of a Staphylococcus-specific chimeric endolysin comprising one or more heterologous domains is an endolysin comprising an Amidase domain of bacteriophage Ply2638, an M23 peptidase domain of lysostaphin (S. simulans) and a cell wall-binding domain of bacteriophage Ply2638. Such Staphylococcus-specific chimeric endolysin is a preferred endolysin according to the invention and is extensively described in WO2012/150858, which is herein incorporated by reference in its entirety. Other preferred endolysins according to the invention are extensively described in WO2013/169104, which is herein incorporated by reference in its entirety. Other preferred endolysins according to the invention are extensively described in WO2017/046021, which is herein incorporated by reference in its entirety.

The present invention may conveniently be combined with the embodiments and endolysins of WO2016/142445, which is herein incorporated by reference in its entirety. In WO2016/142445, intracellular Staphylococcus aureus infections are combatted using an agent the increases the intracellular pH combined with an endolysin that is capable of entry into the cell. Such endolysins that are capable of entering the cell are preferred endolysins according to the invention.

A further preferred endolysin according to the invention is one selected from the group consisting of the endolysins depicted as SEQ ID NO: 19 to SEQ ID NO: 75 in Table 1.

Preferably, an endolysin domain according to the invention has at least 80%, 81%, 82%, 83%, 84%, 85%, 86%, 87%, 88%, 89%, 90%, 91%, 92%, 93%, 94%, 95%, 96%, 97%, 98%, 99% or more preferably 100% sequence identity with a domain depicted in WO2012/150858, WO2013/169104, WO2016/142445 or in any of SEQ ID NO: 3 to SEQ ID NO: 18 (see Table 1).

Preferably, an endolysin according to the invention has at least 80%, 81%, 82%, 83%, 84%, 85%, 86%, 87%, 88%, 89%, 90%, 91%, 92%, 93%, 94%, 95%, 96%, 97%, 98%, 99% or more preferably 100% sequence identity with an endolysin depicted in WO2012/150858, WO2013/169104, WO2016/142445 or in any of SEQ ID NO: 1, 2 and SEQ ID NO: 19 to SEQ ID NO: 75 (see Table 1).

The person skilled in the art will comprehend that mixes of different endolysins may be used in the present invention, e.g. a mix comprising two, three or four endolysins according to the invention. In the embodiments of the invention, the subject is a vertebrate, preferably a mammal, more preferably a human.

The type of cancer to be treated by be any type of cancer where oncogenic bacteria are associated with the pathogenesis and oncogenesis of the cancer. A preferred cancer is a mucosa associated cancer such as (skin) lymphoma and gastric cancer. A more preferred cancer is cutaneous T-cell lymphoma (CTCL).

The person skilled in the art will comprehend that treatment with the endolysin according to the invention may conveniently be combined with other drugs, such as other anti-cancer medicaments. The compounds be present in individual compositions or may be present in a single composition. Accordingly, an endolysin according to the invention may be present in a composition further comprising an excipient, preferably a pharmaceutically acceptable excipient. In said composition according to the invention or pharmaceutical composition according to the invention, a further anti-cancer medicament may be present, such as a chemotherapeutic agent and a monoclonal antibody.

The medical treatment according to the invention includes an endolysin specific for an oncogenic bacterium for the manufacture of a medicament for the prevention, delay or treatment of cancer in a subject in need thereof as well as a method for the prevention, delay or treatment of cancer in a subject in need thereof, comprising administration of an endolysin specific for an oncogenic bacterium to the subject. The embodiments are preferably those as defined here above.

TABLE 1
SEQ ID NO: overview

| SEQ ID NO | Name construct | organism |
|---|---|---|
| 1 | Ply2638 endolysin CDS | Bacteriophage 2638A |
| 2 | Ply2638 endolysin PRT | Bacteriophage 2638A |
| 3 | CWT-LST CDS | S. simulans |
| 4 | CWT-LST PRT | S. simulans |
| 5 | CBD2638 CDS | Bacteriophage 2638A |
| 6 | CBD2638 PRT | Bacteriophage 2638A |
| 7 | CWT-NM3 CDS | S. aureus phage phiNM3 |
| 8 | CWT-NM3 PRT | S. aureus phage phiNM3 |
| 9 | CHAPK CDS | S. phage K |
| 10 | CHAPK PRT | S. phage K |
| 11 | CHAP-ΦTwort CDS | S. phage Twort |
| 12 | CHAP-ΦTwort PRT | S. phage Twort |
| 13 | M23-2638 CDS | Bacteriophage 2638A |
| 14 | M23-2638 PRT | Bacteriophage 2638A |
| 15 | M23-LST CDS | S. simulans |
| 16 | M23-LST PRT | S. simulans |
| 17 | Ami2638 CDS | Bacteriophage 2638A |
| 18 | Ami2638 PRT | Bacteriophage 2638A |
| 19 | CHAPK_CHAPK_CWT-LST CDS | artificial construct |
| 20 | CHAPK_CHAPK_CWT-LST PRT | artificial construct |
| 21 | M23-LST_M23-LST_CWT-LST CDS | artificial construct |
| 22 | M23-LST_M23-LST_CWT-LST PRT | artificial construct |
| 23 | Ami2638_ami2638_CWT-LST CDS | artificial construct |
| 24 | Ami2638_ami2638_CWT-LST PRT | artificial construct |
| 25 | HXaAmi2638_CBD2638 CDS | artificial construct |
| 26 | HXaAmi2638_CBD2638 PRT | artificial construct |
| 27 | HXaAmi2638_CWT-LST CDS | artificial construct |
| 28 | HXaAmi2638_CWT-LST PRT | artificial construct |
| 29 | HXaAmi2638_CWT-NM3 CDS | artificial construct |
| 30 | HXaAmi2638_CWT-NM3 PRT | artificial construct |
| 31 | HXaCHAPK_CBD2638 CDS | artificial construct |
| 32 | HXaCHAPK_CBD2638 PRT | artificial construct |
| 33 | HXaCHAPK_CWT-LST CDS | artificial construct |
| 34 | HXaCHAPK_CWT-LST PRT | artificial construct |
| 35 | HXaCHAPK_CWT-NM3 CDS | artificial construct |
| 36 | HXaCHAPK_CWT-NM3 PRT | artificial construct |
| 37 | HXaCHAPTw_CBD2638 CDS | artificial construct |
| 38 | HXaCHAPTw_CBD2638 PRT | artificial construct |
| 39 | HXaCHAPTw_CWT-LST CDS | artificial construct |
| 40 | HXaCHAPTw_CWT-LST PRT | artificial construct |
| 41 | HXaCHAPTw_CWT-NM3 CDS | artificial construct |
| 42 | HXaCHAPTw_CWT-NM3 PRT | artificial construct |
| 43 | HXaM23-LST_CBD2638 CDS | artificial construct |
| 44 | HXaM23-LST_CBD2638 PRT | artificial construct |
| 45 | HXaM23-LST_CWT-LST CDS | artificial construct |
| 46 | HXaM23-LST_CWT-LST PRT | artificial construct |
| 47 | HXaM23-LST_CWT-NM3 CDS | artificial construct |
| 48 | HXaM23-LST_CWT-NM3 PRT | artificial construct |
| 49 | HXaAmi2638_Ami2638_CBD2638 CDS | artificial construct |
| 50 | HXaAmi2638_Ami2638_CBD2638 PRT | artificial construct |
| 51 | HXaAmi2638_Ami2638_CWT-LST CDS | artificial construct |
| 52 | HXaAmi2638_Ami2638_CWT-LST PRT | artificial construct |
| 53 | HXaAmi2638_Ami2638_CWT-NM3 CDS | artificial construct |
| 54 | HXaAmi2638_Ami2638_CWT-NM3 PRT | artificial construct |
| 55 | HXaCHAPK_CHAPK_CBD2638 CDS | artificial construct |
| 56 | HXaCHAPK_CHAPK_CBD2638 PRT | artificial construct |
| 57 | HXaCHAPK_CHAPK_CWT-LST CDS | artificial construct |
| 58 | HXaCHAPK_CHAPK_CWT-LST PRT | artificial construct |
| 59 | HXaCHAPK_CHAPK_CWT-NM3 CDS | artificial construct |
| 60 | HXaCHAPK_CHAPK_CWT-NM3 PRT | artificial construct |
| 61 | HXaCHAPTw_CHAPTw_CBD2638 CDS | artificial construct |
| 62 | HXaCHAPTw_CHAPTw_CBD2638 PRT | artificial construct |
| 63 | HXaCHAPTw_CHAPTw_CWT-LST CDS | artificial construct |
| 64 | HXaCHAPTw_CHAPTw_CWT-LST PRT | artificial construct |
| 65 | HXaCHAPTw_CHAPTw_CWT-NM3 CDS | artificial construct |
| 66 | HXaCHAPTw_CHAPTw_CWT-NM3 PRT | artificial construct |
| 67 | HXaM23-LST_M23-LST_CBD2638 CDS | artificial construct |
| 68 | HXaM23-LST_M23-LST_CBD2638 PRT | artificial construct |
| 69 | HXaM23-LST_M23-LST_CWT-LST CDS | artificial construct |
| 70 | HXaM23-LST_M23-LST_CWT-LST PRT | artificial construct |
| 71 | HXaM23-LST_M23-LST_CWT-NM3 CDS | artificial construct |
| 72 | HXaM23-LST_M23-LST_CWT-NM3 PRT | artificial construct |
| 73 | M23-LST_Ami2638_CBD2638 PRT | artificial construct |
| 74 | M23-LST_Ami2638_CBD2638 PRT* | artificial construct |
| 75 | MART-1 peptide | artificial construct |

Uneven SEQ ID NOs: 1-71 represent the coding sequences (CDS) of even SEQ ID NOs: 2-72 that represent the respective polypeptide (PRT) sequences.

Definitions

“Sequence identity” is herein defined as a relationship between two or more amino acid (peptide, polypeptide, or protein) sequences or two or more nucleic acid (nucleotide, polynucleotide) sequences, as determined by comparing the sequences. In the art, “identity” also means the degree of sequence relatedness between amino acid or nucleotide sequences, as the case may be, as determined by the match between strings of such sequences. “Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole SEQ ID NO as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

A “nucleic acid molecule” or “polynucleotide” (the terms are used interchangeably herein) is represented by a nucleotide sequence. A “polypeptide” is represented by an amino acid sequence.

A “nucleic acid construct” is defined as a nucleic acid molecule which is isolated from a naturally occurring gene or which has been modified to contain segments of nucleic acids which are combined or juxtaposed in a manner which would not otherwise exist in nature. A nucleic acid molecule is represented by a nucleotide sequence. Optionally, a nucleotide sequence present in a nucleic acid construct is operably linked to one or more control sequences, which direct the production or expression of said peptide or polypeptide in a cell or in a subject.

“Operably linked” is defined herein as a configuration in which a control sequence is appropriately placed at a position relative to the nucleotide sequence coding for the polypeptide of the invention such that the control sequence directs the production/expression of the peptide or polypeptide of the invention in a cell and/or in a subject. “Operably linked” may also be used for defining a configuration in which a sequence is appropriately placed at a position relative to another sequence coding for a functional domain such that a chimeric polypeptide is encoded in a cell and/or in a subject.

“Expression” is construed as to include any step involved in the production of the peptide or polypeptide including, but not limited to, transcription, post-transcriptional modification, translation, post-translational modification and secretion.

A “control sequence” is defined herein to include all components which are necessary or advantageous for the expression of a polypeptide. At a minimum, the control sequences include a promoter and transcriptional and translational stop signals. Optionally, a promoter represented by a nucleotide sequence present in a nucleic acid construct is operably linked to another nucleotide sequence encoding a peptide or polypeptide as identified herein.

The term “transformation” refers to a permanent or transient genetic change induced in a cell following the incorporation of new DNA (i.e. DNA exogenous to the cell). When the cell is a bacterial cell, as is intended in the present invention, the term usually refers to an extrachromosomal, self-replicating vector which harbors a selectable antibiotic resistance.

An “expression vector” may be any vector which can be conveniently subjected to recombinant DNA procedures and can bring about the expression of a nucleotide sequence encoding a polypeptide of the invention in a cell and/or in a subject. As used herein, the term “promoter” refers to a nucleic acid fragment that functions to control the transcription of one or more genes or nucleic acids, located upstream with respect to the direction of transcription of the transcription initiation site of the gene. It is related to the binding site identified by the presence of a binding site for DNA-dependent RNA polymerase, transcription initiation sites, and any other DNA sequences, including, but not limited to, transcription factor binding sites, repressor and activator protein binding sites, and any other sequences of nucleotides known to one skilled in the art to act directly or indirectly to regulate the amount of transcription from the promoter. Within the context of the invention, a promoter preferably ends at nucleotide −1 of the transcription start site (TSS).

A “polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules.

The sequence information as provided herein should not be so narrowly construed as to require inclusion of erroneously identified bases. The skilled person is capable of identifying such erroneously identified bases and knows how to correct for such errors.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a product or a composition or a nucleic acid molecule or a peptide or polypeptide of a nucleic acid construct or vector or cell as defined herein may comprise additional component(s) than the ones specifically identified; said additional component(s) not altering the unique characteristic of the invention. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

### EXAMPLE 1

A subject suffering from cutaneous T-cell lymphoma (CTCL) is treated by topical administration of an endolysin according to the invention specific for Staphylococcus aureus. As a result, STAT3 activation and IL-10 and IL-17 expression are efficiently reduced, thereby treating the cancer.

### EXAMPLE 2

Analysis of the effect of Staphylococcus aureus alpha-toxin on malignant T-cells and non-malignant T-cells such as cytotoxic T-cells.

### SUMMARY

It was demonstrated for the first time that the Staphylococcus aureus alpha-toxin inhibits the proliferation of non-malignant T-cells, such as cytotoxic T-cells, but not of malignant T-cells. In addition, it was demonstrated that Staphylococcus aureus alpha-toxin inhibits cytotoxic activity of CTCL-specific cytotoxic CD8 T-cells. This implicates that the killing of cancer cells by cytotoxic T-cells is inhibited by Staphylococcus aureus alpha-toxin resulting in escape of tumour cells from killing and that Staphylococcus aureus alpha-toxin favours growth and survival of tumour cells. It follows that treatment targeting the alpha-toxin producing Staphylococcus aureus of patients with T cell lymphoma effectively treats the CTCL.

### Introduction

To investigate the role of Staphylococcus aureus alpha-toxin in T-cell lymphoma, it was investigated what the influence of Staphylococcus aureus alpha-toxin on proliferation of non-malignant T-cells, such as cytotoxic T-cells, and of malignant T-cells is; specifically whether there is a difference in proliferation in the presence of Staphylococcus aureus alpha-toxin.

Proliferation of patient-derived malignant and non-malignant CTCL cell lines was measured in the presence and absence of Staphylococcus aureus alpha-toxin by a standard ³H-Thymidine incorporation assay. In addition, a CTL assay (LDH, lactate dehydrogenase) release assay was performed using MART-1 specific CD8 T-cells as effector cells and Mac1 cells pulsed with MART-1 peptide (ELAGIGILTV: SEQ ID NO: 75) as target cells, in the presence and absence of Staphylococcus aureus alpha-toxin.

### Materials and Methods

Proliferation Assay

Malignant T-cell line MyLa2059 cells (Acc No: RRID:CVCL_5342 (Woetmann et al, 2007)) were plated on 96 well plates at a cell density of 1,250 cells per well, while non-malignant T-cell line MyLa1850 cells (Acc No: RRID:CVCL_M415 (Woetmann et al, 2007)), were plated at a cell density of 2,500 cells per well. Cells were cultured at 37° C. in 20 μl medium RPMI-1640 at 5% CO₂ as described in Levring et al, 2015, while being exposed to 2-fold increasing concentrations of alpha-toxin (List lab, Cat. No. #120). After 72 hrs of incubation at 37° C., 1 μCi/well Thymidine [Methyl-³H] (PerkinElmer, Cat. No. NET027250UC) was added for an additional 24 hrs. After harvesting the 96 well plate onto a filter plate using a FilterMateHarvester (PerkinElmer), the filter plate was dried overnight; subsequently 20 μl MicroScint™-20 cocktail (PerkinElmer, Cat No. 6013621) to each well on the counting filter. The filter was then analysed with a TopCount®NXT™ Microplate Scintillation and Luminescence Counter (Packard), counting the incorporated thymidine as counts per minutes (cpm), indirectly reflecting cell proliferation. Counts were normalized to the toxin untreated sample within each group.

CTL Assay

For the cytotoxicity assay, CD8 effector cells (Sorensen et al, 2009) were pre-treated with 1.5 μg/ml alpha-toxin (Sigma, Cat. No. H9395-0.5 MG) for 2 hrs at, while Mac1 target cells (Acc No: RRID:CVCL_H631 (Davis et al, 1992)) were pulsed with 1 μM of MART-1 peptide at 37° C. Cells were plated in phenol free X-VIVO (Lonza, Cat. No. BE02-061Q) media with effector target ratios of 1:4, 1:2 and 1:1. LDH release was quantified from culture supernatants after 4 hrs in co-culture following the manufactures instructions (TaKaRa, Cat. No. MK401). Specific cell lysis (specific cytotoxicity) was calculated after subtracting the background release of the CD8⁺ T cells using the following formula:

Control:
Un-pulsed Mac1 cells→exposed to the respective amount of DMSO as the peptide pulsed cells

Background Subtraction:
Background release of CD8 was subtracted prior to applying the equation below
Formula for calculation of specific cytotoxicity:

$$\% Cytotoxicity = \frac{( Experimental release - spontaneous release )}{( Maximum release - spontaneous release )} \times 100$$

Experimental release: Co-culture of pulsed Mac1 cells with effector cells
Spontaneous release: Co-culture of DMSO Mac1 cells with effector cells
Maximum release: Mac1 cells treated with Tritox-100 X

The spontaneous release was measured in co-cultures of CD8+ T cells with un-pulsed targets cells and the maximum release in monocultures of effector cells in the presence of Triton X-100 (Sigma-Aldrich, #T8787).

### Results and Conclusion

Proliferation Assay

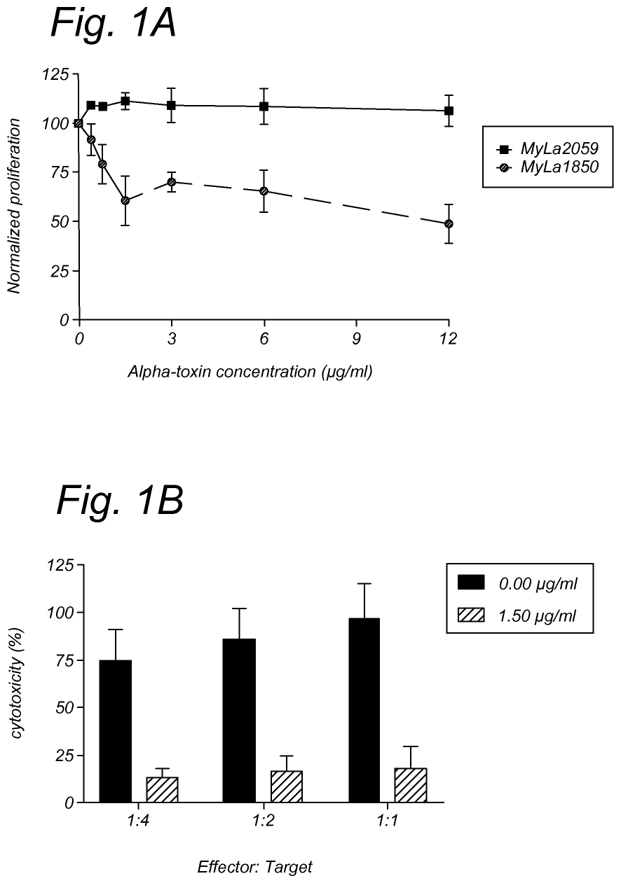

The results of the proliferation assay are depicted in FIG. 1A. It is clear that proliferation of malignant MyLa3059cells is not inhibited by Staphylococcus aureus alpha-toxin while proliferation of non-malignant MyLa1850 cells is significantly inhibited by Staphylococcus aureus alpha-toxin.

CTL Assay

The results of the CTL assay are depicted in FIG. 1B. It is clear that the CD8 effector cells effectively kill the MART-1 peptide pulsed Mac1 target cells. In contrast, when CD8-effector cells have been pre-incubated with Staphylococcus aureus alpha-toxin, cytotoxic activity is severely hampered.

Altogether, it has clearly been demonstrated that Staphylococcus aureus alpha-toxin inhibits killing of cancer cells by CD8 cytotoxic T-cells. In addition, it has been demonstrated that proliferation of malignant cells is not inhibited by Staphylococcus aureus alpha-toxin while proliferation of non-malignant T-cells is inhibited by Staphylococcus aureus alpha-toxin, providing a selective advantage to malignant T-cells, resulting in outgrowth of malignant cells at the expense of non-malignant cells. These results provide a clear incentive to treat CTCL, apart from state of the art anti-cancer medication, with an agent, such as an endolysin, that specifically eradicates Staphylococcus aureus, while leaving other, commensal, microorganisms intact.

### REFERENCES

Woetmann, A. et al. Nonmalignant T cells stimulate growth of T-cell lymphoma cells in the presence of bacterial toxins. Blood 109, 3325-3332 (2007).

Davis, T. H., Morton, C. C., Miller-Cassman, R., Balk, S. P. & Kadin, M. E. Hodgkin's disease, lymphomatoid papulosis, and cutaneous T-cell lymphoma derived from a common T-cell clone. N. Engl. J. Med. 326, 1115-1122 (1992).

Levring TB1, et al. Human CD4+ T cells require exogenous cystine for glutathione and DNA synthesis. Oncotarget. 2015 Sep. 8; 6(26):21853-64.).

Sorenson, K et al., The immunodominant HLA-A2-restricted MART-1 epitope is not presented on the surface of many melanoma cell lines Cancer Immunol Immunother (2009) 58:665-675.

1. An endolysin specific for an oncogenic bacterium for use in the prevention, delay or treatment of cancer in a subject in need thereof.

2. An endolysin specific for an oncogenic bacterium for use according to claim 1, wherein the oncogenic bacterium is a Staphylococcus, preferably a Staphylococcus aureus, more preferably an antibiotic resistant Staphylococcus aureus, such as a methicillin resistant Staphylococcus aureus (MRSA).

3. An endolysin specific for an oncogenic bacterium for use according to claim 1, wherein the endolysin is a Staphylococcus-specific endolysin.

4. An endolysin specific for an oncogenic bacterium for use according to claim 3, wherein the endolysin is a recombinant Staphylococcus-specific endolysin, preferably a recombinant Staphylococcus-specific chimeric endolysin comprising one or more heterologous domains.

5. An endolysin specific for an oncogenic bacterium for use according to claim 4, wherein the recombinant Staphylococcus-specific endolysin is selected from the group consisting of the endolysins depicted as SEQ ID NO: 19 to SEQ ID NO: 75 in Table 1.

6. An endolysin specific for an oncogenic bacterium for use according to claim 1, wherein the subject is a human.

7. An endolysin specific for an oncogenic bacterium for use according to claim 1, wherein the cancer is a mucosa associated cancer such as (skin) lymphoma and gastric cancer.

8. An endolysin specific for an oncogenic bacterium for use according to claim 1, wherein the cancer is cutaneous T-cell lymphoma (CTCL).

9. An endolysin specific for an oncogenic bacterium for use according to claim 1, wherein the endolysin is present in a composition further comprising an excipient, preferably a pharmaceutically acceptable excipient.

10. An endolysin specific for an oncogenic bacterium for use according to claim 9, wherein the composition further comprises another anti-cancer medicament.

11. A method for the prevention, delay or treatment of cancer in a subject in need thereof, comprising administration of an endolysin specific for an oncogenic bacterium to the subject.

12. Use of an endolysin specific for an oncogenic bacterium for the manufacture of a medicament for the prevention, delay or treatment of cancer in a subject in need thereof.
